(19) United States
(12) Patent Application Publication
(10) Pub. No.: US 2016/0030542 A1
(43) Pub. Date: Feb. 4, 2016

(54) Toxoid, Compositions and Related Methods

(71) Applicant: SANOFI PASTEUR, INC., Swiftwater, PA (US)

(72) Inventors: Mark Shieh, Swiftwater, PA (US); Mike Soika, Swiftwater, PA (US)

(21) Appl. No.: 14/776,189

(22) PCT Filed: Mar. 14, 2014
(86) PCT No.: PCT/US14/29070

§ 371 Date: Sep. 14, 2015

Related U.S. Application Data
(60) Provisional application No. 61/793,376, filed on Mar. 15, 2013.

(51) Int. Cl. A61K 39/08; C12N 9/10

(52) U.S. Cl. CPC A61K 39/08; C12N 9/1051; C12Y 204/01

(57) ABSTRACT

Methods of producing purified clostridial toxin comprising tangential flow filtration, hydrophobic interaction chromatography and anion exchange chromatography are disclosed. These methods provide good yields of C. difficile toxin having a purity of about 90% or greater. Highly purified Clostridial toxins, toxoids (e.g., prepared by inactivating the toxin as disclosed herein) and compositions comprising these toxins and/or toxoids are also disclosed. Methods of using the purified toxins and/or toxoids for example, to elicit an immune response against Clostridium (e.g., C. difficile) are also disclosed.

### RELATED APPLICATIONS

This application claims priority to U.S. Ser. No. 61/793,376 filed Mar. 15, 2013 which is hereby incorporated in its entirety into this application.

### FIELD OF DISCLOSURE

This disclosure relates generally to the field of protein purification. More specifically, it relates to purified clostridial toxins, toxoids, compositions comprising purified toxins or toxoids and methods of preparing purified toxins and toxoids.

### BACKGROUND OF THE DISCLOSURE

Clostridial toxins prepared from culture filtrates require extensive purification to separate the various cellular components (e.g., cellular DNA, cellular proteins, media components) present in the filtrate in order to be suitable for in vivo use. Indeed, a number of purification techniques have been developed for this purpose. Toxins prepared in accordance to these techniques may still however contain appreciable amounts of in-process impurities. Various attempts have been made to purify C. difficile Toxin A and Toxin B from culture filtrates.

In one process, culture filtrates were purified by ultrafiltration, ion-exchange chromatography and acetic acid precipitation. By this process, Toxin B could only be partially purified as evidenced by the inability to distinguish the toxin from several contaminating proteins when analyzed by polyacrylamide gel electrophoresis (PAGE). [Rothman, S. W., et al. Curr. Microbiol. 1981, 6:221-224; Sullivan, N. M., et al., Infect. Immun., 1982, 35:1032-1040]. A later modification of this process involved the purification of toxins from dialyzed filtrates of C. difficile by hydrophobic interaction chromatography and ion-exchange chromatography (Rothman S. W., Infect. Immun., 1984, p. 324-331). However, this process too was unable to provide highly purified forms of both Toxin A and Toxin B.

A method of co-purifying Toxins A and B from C. difficile culture filtrate using diafiltration, ammonium sulfate precipitation and gel chromatography (S-300 Sephacryl size-exclusion column) is described in U.S. Pat. No. 6,969,520. Toxins prepared by this method had a purity of 50% to 60% (or less, e.g., 44%) and included a number of impurities (for example, an about 35 kDa impurity, C. difficile 3-hydroxy butryl CoA dehydrogenase, a 45-47 kDa impurity (C. difficile glutamate dehydrogenase), and a 60-70 kDa protein (a homologue of groEL or the bacterial hsp60 family of proteins)).

Currently available methods are not suitable for large scale production. Accordingly, there is a need in the art for alternative methods of preparing purified toxins. Methods for providing purified C. difficile toxins in large scale are provided by this disclosure.

### SUMMARY OF THE DISCLOSURE

This disclosure provides methods for preparing highly purified toxins and toxoids. C. difficile toxins and toxoids are obtained in a purified form and in sufficient yields by using the methods described herein. In some embodiments, this disclosure provides methods for the production of C. difficile toxin by purifying toxin from an impure aqueous solution by a combination of hydrophobic interaction chromatography, and anion exchange chromatography, optionally in the presence of certain excipients. In certain embodiments, the methods may include anion exchange chromatography using a non-polysaccharide based support. In some embodiments, the methods may also include hydrophobic interaction chromatography using a hydrophobic interaction support with attached phenyl, butyl or propyl groups. The impure aqueous solution may in some embodiments be subjected to ultrafiltration and/or diafiltration steps (e.g., by tangential flow filtration). The methods described herein provide toxins at a purity level of about >90% (e.g., about 90%, about 94%, about 95-97%, about 98%, about 99% or greater) (e.g., “substantially free of impurities”). The purified toxin(s) may be inactivated (e.g., to produce “toxoid(s)”) using chemical agents or other methods that are available to those of ordinary skill in the art (such as e.g., but not limited to, formaldehyde, glutaraldehyde or beta-priopiolactone). Highly purified toxins and toxoids and compositions comprising such toxins or toxoids are also provided. Such highly purified components (and related compositions) may be used to protect subjects from and/or treat subjects with symptomatic C. difficile infection (e.g., as an immunological composition and/or vaccine). Exemplary methods, toxins, toxoids, compositions thereof, and uses therefore may include, but are not limited to those set out in the Examples. Other embodiments will be clear to those of ordinary skill in the art from this disclosure.

### BRIEF DESCRIPTION OF THE DRAWINGS

FIG. 1. Exemplary toxin purification process.

FIG. 2. Immunogenicity of toxoids.

### DETAILED DESCRIPTION

The disclosure provides methods for preparing highly purified toxins and toxoids. Of particular interest herein are C. difficile Toxins A and/or B and/or derivatives thereof (e.g. genetically detoxified versions, truncated forms, fragments, and the like). For the purposes of this disclosure, Toxin A and/or Toxin B may include any C. difficile toxin that may be identified as Toxin A and/or Toxin B using standard techniques in the art. Exemplary techniques may include, for instance, immunoassays such as ELISA, dot blot or in vivo assays. Reagents useful in making such identifications may include, for instance, anti-Toxin A rabbit polyclonal antisera (e.g., Abcam® Product No. ab35021 or Abcam® Product No. ab93318) or an anti-Toxin A mouse monoclonal antibody (e.g., any of Abcam® Product Nos. ab19953 (mAb PCG4) or ab82285 (mAb B618M)), anti-Toxin B rabbit polyclonal antisera (e.g., Abcam® Product No. ab83066) or an anti-Toxin B mouse monoclonal antibody (e.g., any of Acam® Product Nos. ab77583 (mAb B428M), ab130855 (mAb B423M), or ab130858 (mAb B424M)) (all available from Abcam® (Cambridge, Mass.)).

Methods are provided herein that are scalable from analytical to production scale. In some embodiments, the methods provide toxins at a purity level of about 90% or greater (e.g., about 90%, about 95-97%, about 98%, about 99% or greater). In some embodiments, compositions comprising toxins and/or toxoids of such purity levels may be considered “substantially free of impurities”. Methods for inactivating the purified toxins into toxoids are also provided. Methods for using such toxins and toxoids are also provided. These methods, toxins, toxoids, compositions, and methods for using the same are provided herein.

The methods described herein typically involve the purification of C. difficile toxin from an impure aqueous solution of C. difficile toxin. The method is applicable to toxins from virtually any strain of C. difficile. Preferred strains of C. difficile are strains which produce Toxin A and/or B and may be, for example, strains of toxinotype 0 (e.g., VPI10463/ATCC43255, 630), III (e.g., 027/NAP/B1), V (e.g., 078) and VIII (e.g., 017).

Typically, C. difficile is grown in a fermentor under controlled conditions until the desired concentration of cells as determined by OD measurement is reached. The fermentor broth is harvested and clarified by removing the majority of cells and cell debris impurities by filtration (e.g., using membrane filters). Filtration may be performed using filters such as depth filters (e.g., high performance depth filters such as, e.g., Pall Stax). Filtered broth may then be sterilized by microfiltration, preferably using membrane filters of about 0.2 μm pore size. The resulting broth filtrate typically includes C. difficile toxin (e.g, Toxin A and/or Toxin B) and other impurities, representing an impure aqueous solution of C. difficile toxin. To purify (or substantially purify) the C. difficile toxin, methods comprising multiple stages (e.g., steps) are provided.

In some embodiments, the first stage may include the concentration, ultrafiltration, and/or diafiltration of broth containing C. difficile toxin. The concentration, ultrafiltration and diafiltration may be performed by tangential flow filtration (TFF) (e.g., cross-flow filtration). Concentration may generally be carried out by ultrafiltration on a membrane, having a suitable cutoff threshold (e.g., between about 100 kDa and about 400 kDa). TFF is well-known to those of skill in the art and equipment and protocols for its implementation are commercially available from a variety of manufacturers including but not limited to the Pall Corporation (Port Washington, N.Y; www.pall.com) and EMD Millipore (Billerica, Mass.; www.millipore.com). These methods may be implemented using any of several widely-available systems such as those comprising flat sheet membranes or hollow-fiber membranes. Exemplary membranes may include, for instance, Spectrum membranes and Biomax membranes by Millipore. In large scale production, flat sheet systems with the ability to prevent excessive shear forces on the toxins (e.g., having an open flow channel) may be used. Tangential ultrafiltration may be conducted using membranes in the form of flat sheets and having an appropriate cutoff threshold (e.g., about any of 50-100 kDa).

The filtrate (e.g., produced as described above) may be diafiltered into a suitable buffer such as, for example, a Tris buffer (e.g., about any of 25 mM to 50 mM), optionally including a salt (e.g., 25 mM-50 mM NaCl), EDTA (e.g., 0.2 mM), having a pH of about 7.0 to about 8.0 (e.g., pH 7.5). The buffer may also include dithiothreitol (DTT) but this may not always be necessary and/or advised. In some preferred embodiments, then, DTT is specifically excluded as it may have a negative effect on process outcome. The diafiltered product may be then filtered one or more times using an appropriate system (e.g., a filter capsule comprising a 0.8 μm membrane and 0.2 μm membrane (e.g., a Sartorius, Pall EKV, or a Millipore filter)).

The diafiltrate may then be subjected to hydrophobic interaction chromatography (HIC) to remove hydrophilic and slightly hydrophobic impurities. The principles of HIC are well known in the art. Briefly, and without being bound by any particular mechanism of action, HIC is based on the interaction between hydrophobic groups on a protein and a hydrophobic ligand on the applicable solid support. The adsorption of hydrophobic groups on a protein to the hydrophobic ligands on the support may be promoted by the addition of lyotrophic salts (such as, e.g., but not limited to ammonium sulfate, sodium sulfate). Desorption of bound solutes may be achieved by stepwise or gradient elution with buffers of low salt concentration. The binding of proteins to the hydrophobic support may be affected by a number of factors including: (i) the type of ligand, particularly its hydrophobicity (e.g., ether, phenyl, butyl, or propyl); (ii) the ligand density of the solid support; (iii) the backbone material of the matrix; (iv) the hydrophobic nature of the protein; and, (v) the type of salt used. A column that is less hydrophobic will typically require a higher salt concentration for binding. To promote hydrophobic interactions, ammonium sulfate may be added to the diafiltrate solution (e.g., to an appropriate final concentration (e.g., about any of 0.4-1.2M or about 0.4 to about 0.9M, such as about any of 0.4, 0.5, 0.6, 0.7, 0.8, 0.9, 1.0, 1.1, or 1.2M)). The solution may be chromatographed on a hydrophobic interaction support (e.g., resin or membrane) such as Sepharose (with e.g., an attached phenyl, butyl, ether or propyl ligand, preferably butyl, ether or propyl). In certain most preferred embodiments, the support may include a butyl ligand (e.g., butyl S Sepharose). The use of such chromatographic supports may provide for elution of the toxin from the support without use of solvent (e.g., isopropanol (IPA)) and without the addition of DTT (see, e.g., the Examples). As shown in the Examples, HIC using a butyl Sepharose (e.g., Butyl S HIC Sepharose) may provide for increased recovery of toxins and elution of toxins A and B without the use of solvent (e.g., IPA).

Following column equilibration, the solution can be directly loaded on the column. A loading buffer may be used to allow for the binding of toxin to the column support material used. Suitable loading buffers may comprise, for example, a suitable buffering component such as, for example, Tris at a suitable concentration (e.g., at any of about 15 mM to 50 mM, e.g. about any of 15, 20 25, 30, 35, 40, 45 or 50 mM, preferably about 25 mM) at a typical pH of about 7.0 to about 8.0 (preferably, pH 8.0), with an appropriate amount of of salt (e.g., 0.8M-1.0M (NH₄)₂S0₂, such as about any of 0.8, 0.9, or 1.0M, preferably about 0.9M) Many other salts may also be appropriate such as, for instance, those typically used in HIC such as sodium sulfate and/or NaCl (e.g., 25 mM to 50 mM NaCl such as about any of 25, 30, 35, 40, 45 or 50 mM NaCl), as would be understood by those of ordinary skill in the art. Other components such as EDTA (e.g., about 0.2 mM) may also be included. DTT may or may not also be included (e.g., as it may affect toxin autoproteolytic activity). As mentioned above, DTT is preferably excluded (thus, the process is preferably carried out without including and/or in the absence of DTT).

Column loading may typically be followed by a wash step using an appropriate buffer comprising a suitable buffering component such as, for example, Tris at a suitable concentration (e.g., about 15 mM to about 50 mM Tris (e.g. about any of 15, 20 25, 30, 35, 40, 45 or 50 mM, preferably about 25 mM Tris; 0.8M-1.0M ammonium sulfate (preferably 0.9M)), pH about 7.5-8.0, (e.g., about any of pH 7.5, 7.6, 7.7, 7.8, 7.9 or 8.0)) to wash away impurities. Bound toxins may then be eluted in one or more fractions using a suitable buffer (e.g., 10-40 mM Tris (e.g. about any of 10, 15, 20, 25, 30, 35, or 40 mM Tris, preferably about 25 mM Tris), pH 7.5-8.0 (e.g., about any of pH 7.5, 7.6, 7.7, 7.8, 7.9 or 8.0)). The conductivity (ionic strength) of the eluted toxin fractions may then be lowered to an appropriate level (e.g., 6-8 mS/cm (conductivity units) or less, such as, e.g., any of about 6, 6.5, 7.0, 7.5 or 8.0 mS/cm) using an appropriate diluent such as, for example, water for injection (WFI).

The C. difficile toxins (in eluate) may then be further purified by anion exchange chromatography (AEX). The principles of AEX are well known in the art. Without being bound by any particular mechanism of action, this method typically relies on the charge-charge interactions between the solutes/molecules to be isolated and the charge on the anion exchange support material (e.g., resin, membrane) used. As the toxins are negatively charged at physiological pH ranges, the support may contain immobilized positively charged ion-exchange groups/moieties. The positively charged moieties are generally quaternary amino groups or diethylaminoethane (DEAE) groups. Thus, the eluted aqueous solution may be allowed to come into contact with an anion exchange support (e.g., resin, membrane) that has positively charged ion exchange groups under conditions which allow the toxins to bind to the support. Although AEX may be performed using polysaccharide-based supports (such as, e.g., DEAE-Sepharose resin, GE (Q, DEAE, DEAP) Sepharose resin, and/or Sartobind membranes), it was discovered here that non-polysaccharide based supports (e.g., synthetic polymers, and inorganic materials) improved toxin purity and yield. Suitable exemplary membranes that may be used include those made of, for instance, microporous polyethersulfone (PES) chemically modified with polymers that are crosslinked to produce quaternary amine surfaces having positively charged ion-exchange groups (such as e.g., membranes by Pall or Natrix). For instance, exemplary resins suitable for use include methacrylate-based resins to which quaternary amine functional groups are attached (such as e.g., Tosoh Super Q resin (Toso Biosciences), Bio-Rad Q resin (Bio-Rad)). As shown in the Examples, such chromatographic supports (resins, membranes) may provide for the efficient and/or improved separation of alcohol dehydrogenase (ADH) impurity from toxins as compared to conventional techniques.

The AEX chromatographic support may be equilibrated with an appropriate buffer (e.g., 25 mM Tris, 0 mM MgCl₂). Following column equilibration, the solution comprising toxin(s) may be directly loaded on the column. A loading buffer having a conductivity and/or pH that allows for the binding of toxin to the column support is typically used. Toxin A and Toxin B may be individually eluted using appropriate buffers (e.g., of increasing ionic strength) having an appropriate salt concentration. For instance, Toxin A may be eluted with a buffer of a lower salt concentration as compared to Toxin B. In some embodiments, toxin A may be eluted with a low salt buffer comprising one or more sodium and/or magnesium salts such as, for example, about 2 to about 32 mM MgCl₂, preferably about 27 mM MgCl₂, or about 125 to about 160 mM, or about 140 mM (e.g., 141 mM) NaCl. Toxin B may be eluted with a high salt buffer comprising one or more sodium and/or magnesium salts such as, for example, about 120 to about 150 mM, or about 122 to about 148 mM MgCl₂, preferably about 135 mM MgCl₂, or about 450 to about 550 mM, or about 500 mM (e.g., 488 mM)) NaCl. Impurities may be typically eluted at a salt concentration between that of the high salt and the low salt buffer (e.g., 65-79 mM MgCl₂, typically about 72 mM MgCl₂). Many different components may be suitable for use in buffering (e.g., stabilizing the pH) such as, for example, Tris at a suitable amount (e.g., about 25 to about 50 mM). Other buffers and salts may also be used, as would be understood by those of ordinary skill in the art. In some embodiments, elution from AEX support using MgCl₂ is preferred where toxin concentration is high and/or where aqueous solution of toxins includes certain impurities (e.g., ADH, hsp60). As shown in the Examples below, such a situation may be encountered when C. difficile is cultured using a media supplemented with sorbitol.

Chromatography is typically carried out in the presence of aqueous buffer systems to avoid protein denaturation. For instance, any conventional buffers such as Tris, Tricine, phosphate (PBS), glycylglycine, MES, MOPS, HEPES, bis-tris-propane-HCl, or others may be appropriate for AEX. Positively charged buffering ions should be used on anion exchangers to avoid an interaction or binding to the functional group. In some /embodiments, for example, Tris (pKa 8.2) may be used with Cl⁻ as the counterion.

Preferably, purified (or substantially purified) Toxin A and/or purified (or substantially purified) Toxin B may be concentrated and diafiltered into an appropriate storage buffer (e.g., a citrate, phosphate, glycine, carbonate, bicarbonate buffer). Reduced toxin degradation has been observed using citrate buffers, which may therefore be preferred (see, e.g., the Examples).

The final purified toxin is generally at least about any of 80%, 85%, 90%, 95%, 97.5%, 98%, 99%, or 100% purified), as measured using standard techniques (e.g., Capillary Gel Electrophoresis and/or SDS/PAGE assay). As shown in the Examples, the methods described here may be used to produce Toxin A purified to about 99% and Toxin B to about 98%.

The methods described herein also provide enhanced scalability, process control, product purity and product stability. To increase scale, for instance, columns of increased dimensions may be used and the flow rates may be adjusted accordingly.

The methods result in a good yield of C. difficile toxin with limited impurities and sufficient potency/cytotoxicity. It has been found, for instance, that purification by the methods described herein (e.g., using hydrophobic interaction chromatography and/or anion exchange chromatography with a non-polysaccharide based support) removes a large amount of contaminants (e.g., biomolecules, including DNA, lipids and proteins) from culture filtrate. It is noted that AEX may be conducted before or after hydrophobic interaction chromatography (HIC). For instance, HIC may be performed before AEX, or AEX may be performed before HIC. AEX may also be repeated as desired by the user in order to further improve purity levels.

The purified toxins may be inactivated (e.g., to produce toxoids) using techniques known to those of ordinary skill in the art such as, for example, by using chemical agents (e.g., but not limited to, formaldehyde, glutaraldehyde or B-priopiolactone). For instance, the toxins may be inactivated using formaldehyde. The purified toxins may be mixed at an applicable target Toxin A: Toxin B ratio (e.g., 3:1, 3:2) and then inactivated or may be inactivated individually. For example, to inactivate toxins, about 4 mg/ml formaldehyde may be added to about 0.5 mg/ml toxin. Inactivation may proceed at an appropriate temperature (e.g., about 2-8° C., about 25° C. or less) and at an appropriate pH (e.g., about pH 7.0) for an appropriate amount of time (e.g., about 18-21 days). Preferred methods of inactivation (e.g., “toxoiding”) include those described in copending U.S. Ser. No. 61/790,423 filed Mar. 15, 2013, which is incorporated-by-reference in its entirety into this application.

The resulting toxoid preparations may be stored in a storage buffer that may prevent reversion of a toxoid into a toxin (such as, for example, but not limited to, citrate, phosphate, glycine, carbonate or bicarbonate) at a pH 8.0 or less (e.g., 6.5-7.5). The buffer preferably includes at least one or more pharmaceutically acceptable excipients that increase the stability of the toxoids and/or delay or decrease aggregation of the toxoids. Excipients of use include for example but are not limited to sugars (e.g., trehalose, sucrose) or sugar alcohols (e.g., sorbitol), salts (sodium chloride, potassium chloride, magnesium chloride, magnesium acetate), formaldehyde (0.001-0.02%), or combinations thereof. Other excipients suitable for use are described in the art, such as WO2009/035707 (US 2011/045025(A1)), which is incorporated herein in its entirety.

Although the toxoid preparations may be mixed directly with storage buffer, the preparations may also be concentrated and diafiltered into an appropriate buffer solution. Preferably, concentration and diafiltration is done using tangential flow filtration to minimize protein shear while ensuring removal of formaldehyde and exchange into storage buffer. An exemplary storage buffer comprises an appropriate amount of citrate (e.g. 20 mM), an appropriate pH (e.g., pH 7.5), at least one sugar at an appropriate amount (e.g., 4% to 8% sucrose, such as 5%), and about 0.016±0.004% formaldehyde (w/v). The concentration of formaldehyde may adjusted if required to maintain target concentration of formaldehyde (e.g., 0.016±0.004% formaldehyde (w/v)) to prevent reversion. In some embodiments, formaldehyde may be present in such compositions at lower levels such as, for instance, 0.008% (w/v) or 0.004% (w/v). Preferred methods of storage include those described in copending U.S. Ser. No. 61/790,423 filed Mar. 15, 2013, which is incorporated-by-reference in its entirety into this application.

The toxoid preparations may also be filtered (e.g., using 0.2 μm membrane filer) to remove small protein aggregates that may affect the protein concentration by adsorbance at 280 nm to allow for formulation of the pharmaceutical composition at the intended toxoid A: toxoid B ratio. Toxoid A and Toxoid B may be combined at the intended Toxoid A: Toxoid B ratio before storage. Compositions of combined or individual toxoids may be stored at for example, 2-8° C. in liquid or lyophilized form (e.g., in which form formaldehyde may be present at, for instance, about 0.016% (w/v)). If in liquid form, the toxoids are preferably stored at <−60° C.

Toxoids may be formulated for use as pharmaceutical compositions (e.g., immunogenic and/or vaccine compositions). For example, compositions comprising the C. difficile toxoids may be prepared for administration by suspension of the toxoids in a pharmaceutically acceptable diluent (e.g., physiological saline) and/or by association of the toxoids with a pharmaceutically acceptable carrier. Such pharmaceutical formulations may include one or more excipients (e.g., diluents, thickeners, buffers, preservatives, adjuvants, detergents and/or immunostimulants) such as those known and / or available to those of ordinary skill in the art. Suitable exicipents are typically compatible with the toxoid and the adjuvant (e.g., in adjuvanted compositions), with examples thereof being known and available to those of ordinary skill in the art. Compositions may be in liquid form, or lyophilized (as per standard methods) or foam dried (as described by, e.g., U.S. Pat. Pub. 2009/110699, which is incorporated into this disclosure in its entirety). As mentioned above, the compositions may also be lyophilized. In some embodiments, lyophilized compositions may be stored between about 2° C. to about 8° C.

To prepare a vaccine for administration, a dried composition may be reconstituted with an aqueous solution such as, for example, water for injection, or a suitable diluent or buffer solution. In certain examples, the diluent may include formaldehyde as described herein. The diluent may include adjuvant (e.g., aluminum hydroxide) with or without formaldehyde. An exemplary diluent may be an aqueous solution of NaCl and aluminum hydroxide. Such a diluent may be used to reconstitute the dried composition. The pharmaceutical compositions may comprise a dose of the toxoids of about 10 to 150 μg/mL (e.g., any of about 10, 20, 30, 40, 50, 60, 70, 80, 90, 100, 110, 120, 130, 140 or 150 μg/mL). Typically, a volume of a dose for injection is about 0.5 mL or 1.0 mL. Dosages may be increased or decreased as necessary to modulate immune response (and / or if side effects are observed) in a subject. The toxoids may be administered in the presence or absence of an adjuvant, in amounts that may be determined by one skilled in the art. Exemplary adjuvants may include, for instance, aluminum compounds, such as aluminum hydroxide, aluminum phosphate and aluminum hydroxyl phosphate.

The vaccine compositions can be administered by the percutaneous (e.g., intramuscular, intravenous, intraperitoneal or subcutaneous), transdermal, and/or mucosal route in amounts and in regimens determined to be appropriate by those skilled in the art to subjects that have, or are at risk of developing, symptomatic C. difficile infection. These populations include, for example, subjects that have received broad spectrum antibiotics, such as hospitalized elderly patients, nursing home residents, chronically ill patients, cancer patients, AIDS patients, patients in intensive care units, and patients receiving dialysis treatment. The vaccine may be administered one, two, three, four 1, 2, 3, 4 or more times. When multiple doses are administered, the doses may be separated from one another by, for example, any of one to six days, one week, two weeks, three weeks, or one or more months (e.g., several months). Thus, this disclosure also provides methods of eliciting an immune response against the toxins, toxoids, and / or infectious organism comprising the same by administering the pharmaceutical compositions to a subject. This may be achieved by administration of the pharmaceutical compositions (e.g., immunogenic compositions and/or vaccines) described herein to the subject to effect exposure of the toxoids to the immune system of the subject. Thus, the immunogenic compositions and/or vaccines may be used to prevent and/or treat symptomatic C. difficile infections.

Thus, this disclosure provides methods for the production of purified C. difficile toxin by applying an impure aqueous solution comprising C. difficile toxin to a hydrophobic interaction support (e.g., HIC) to bind C. difficile toxin thereto; eluting the bound C. difficile toxin from the hydrophobic interaction support; applying the eluted C. difficile toxin to an anion-exchange support (e.g., AEX) selected from the group consisting of: (i) a polymethacrylate resin with quaternary amine functional groups; and (ii) a polyethersulfone membrane with quaternary amine functional groups, to bind C. difficile toxin thereto; and, eluting the bound C. difficile toxin from the anion-exchange support. In some embodiments, the methods further comprise subjecting an impure aqueous solution comprising C. difficile toxin to tangential flow filtration before HIC and/or recovering purified C. difficile toxin from the AEX eluate. The toxin is typically C. difficile toxin A or C. difficile toxin B. In some embodiments, the the aqueous solution comprising C. difficile toxin comprises C. difficile toxin A and C. difficile toxin B which may be individually eluted from the anion-exchange support. In some embodiments, the hydrophobic interaction support for HIC is a matrix with attached phenyl groups, a matrix with attached butyl S groups (e.g., Sepharose matrix), or a matrix with attached propyl groups. In some embodiments, the anion exchange support may be a polymethacrylate resin with bound quaternary amine functional groups, comprise the moiety —O—R—N⁺—(CH₃)₃ wherein R is a polymer; and/or, the anion exchange support is Tosoh Super Q 650M. In some embodiments, the impure aqueous solution of C. difficile toxin is obtained by growing cells of C. difficile in a growth medium to provide a culture broth containing C. difficile toxin and grown cells, and separating the culture broth from the grown cells to provide the impure aqueous solution of C. difficile toxin. In some embodiments, the impure aqueous solution of C. difficile toxin comprises sorbitol. Also provided are purified C. difficile toxins obtained in accordance with any of these methods (e.g., having a purity of about 90% or greater (e.g., about any of 91%, 92%, 93%, 94%, 95%, 96%, 97%, 98%, 99% or 100%)). In some embodiments, the methods may provide a C. difficile toxin A purified to about 92.8%. In some embodiments, the methods may provide a C. difficile toxin B purified to about 91%. In some embodiments, the purified C. difficile toxin may be substantially free of solvent and/or detectable alcohol dehydrogenase. The methods may also comprise inactivating the recovered, purified C. difficile toxin with a chemical agent (e.g., formaldehyde) to provide a C. difficile Toxoid. This disclosure also provides C. difficile toxoids obtained in accordance with any of these methods. The toxoids (e.g., Toxoid A and/or Toxoid B) may also be combined with a pharmaceutically acceptable excipient to provide a pharmaceutical composition (e.g., an immunogenic composition and/or vaccine). In some embodiments, such compositions may comprise C. difficile Toxoid A and C. difficile B at a ratio of 3:2 by weight. This disclosure also provides methods for eliciting an immune response in a subject (e.g., against C. difficile) by administering to the subject any one or more of the compositions provided herein. In some embodiments, the methods comprise preventing or treating symptomatic and/or non-symptomatic C. difficile infection in a subject. The methods may also be used to treat a subject that does not have, but may be at risk of developing symptomatic C. difficile infection.

Although preferred embodiments have been described herein, it is understood that variations and modifications are contemplated and are readily apparent to those skilled in the art.

The terms “about”, “approximately”, and the like, when preceding a list of numerical values or range, refer to each individual value in the list or range independently as if each individual value in the list or range was immediately preceded by that term. The terms mean that the values to which the same refer are exactly, close to, or similar thereto. For instance, “about” or “approximately” may indicate a value within ten percent of the indicated value (e.g., “about 30%” may include anywhere from 27% to 33%).

As used herein, a subject or a host is meant to be an individual. The subject can include domesticated animals, such as cats and dogs, livestock (e.g., cattle, horses, pigs, sheep, and goats), laboratory animals (e.g., mice, rabbits, rats, guinea pigs) and birds. In one aspect, the subject is a mammal such as a primate or a human.

Optional or optionally means that the subsequently described event or circumstance can or cannot occur, and that the description includes instances where the event or circumstance occurs and instances where it does not. For example, the phrase optionally the composition can comprise a combination means that the composition may comprise a combination of different molecules or may not include a combination such that the description includes both the combination and the absence of the combination (i.e., individual members of the combination).

Ranges may be expressed herein as from about one particular value, and/or to about another particular value. When such a range is expressed, another aspect includes from the one particular value and/or to the other particular value. Similarly, when values are expressed as approximations, by use of the antecedent about or approximately, it will be understood that the particular value forms another aspect. It will be further understood that the endpoints of each of the ranges are significant both in relation to the other endpoint, and independently of the other endpoint. Ranges (e.g., 90-100%) are meant to include the range per se as well as each independent value within the range as if each value was individually listed.

When the terms prevent, preventing, and prevention are used herein in connection with a given treatment for a given condition (e.g., preventing infection), it is meant to convey that the treated subject either does not develop a clinically observable level of the condition at all, or develops it more slowly and/or to a lesser degree than he/she would have absent the treatment. These terms are not limited solely to a situation in which the subject experiences no aspect of the condition whatsoever. For example, a treatment will be said to have prevented the condition if it is given during exposure of a subject to a stimulus that would have been expected to produce a given manifestation of the condition, and results in the subject experiencing fewer and/or milder symptoms of the condition than otherwise expected. A treatment can “prevent” symptomatic infection by resulting in the subject displaying only mild overt symptoms of the infection; it does not imply that there must have been no penetration of any cell by the infecting microorganism.

Similarly, reduce, reducing, and reduction as used herein in connection with the risk of symptomatic infection with a given treatment (e.g., reducing the risk of symptomatic C. difficile infection) typically refers to a subject developing symptomatic infection more slowly or to a lesser degree as compared to a control or basal level of developing symptomatic infection in the absence of a treatment (e.g., administration or vaccination using the toxins or toxoids disclosed). A reduction in the risk of symptomatic infection may result in the subject displaying only mild overt symptoms of the infection or delayed symptoms of infection; it does not imply that there must have been no penetration of any cell by the infecting microorganism.

It must also be noted that, as used in this disclosure and the appended claims, the singular forms “a”, “an”, and “the” include plural referents unless the context clearly dictates otherwise.

All references cited within this disclosure are hereby incorporated by reference in their entirety. Certain embodiments are further described in the following examples. These embodiments are provided as examples only and are not intended to limit the scope of the claims in any way.

### EXAMPLES

The following examples are provided solely for purposes of illustration and are not intended to limit the scope of the disclosure. Changes in form and substitution of equivalents are contemplated as circumstances may suggest or render expedient. Although specific terms have been employed herein, such terms are intended in a descriptive sense and not for purposes of limitations. Methods of molecular genetics, protein biochemistry, and immunology used, but not explicitly described in this disclosure and these Examples, are amply reported in the scientific literatures and are well within the ability of those skilled in the art.

### Example 1

This example describes a method for manufacturing purified C. difficile toxins and toxoids. A C. difficile working seed (strain VPI10463/ATCC43255) was used to inoculate preconditioned culture medium comprising soy peptone, yeast extract, phosphate buffer and sodium bicarbonate, pH 6.35-7.45 (SYS medium) and scaled up from a 4 mL Working Cell Bank (WCB) vial to a 160 L culture. Upon reaching the desired density and the 10-12 hour incubation period, the entire 160 L of culture was processed for clarification and 0.2 μm filtration. The culture from one more production fermentor was harvested and subjected to membrane filtration (e.g., using a Meisner membrane filter) to remove C. difficile cells and cell debris impurities. The resulting clarified culture filtrate was concentrated and diafiltered by tangential flow filtration into a Tris buffer with NaCl, EDTA, and DTT. The resulting solution was filtered using a membrane filter, the concentration of ammonium sulfate was increased (e.g., to about 0.4M) and then a further filtration was performed (e.g., using a membrane filter). This aqueous solution contained C. difficile toxin A and toxin B. The aqueous solution was subjected to hydrophobic interaction chromatography.

The C. difficile toxins were bound to a Phenyl FF Sepharose column (GE Phenyl FF Sepharose). The column was washed with 0.2 mM ammonium sulphate in Tris buffer and two fractions of the C. difficile toxins were eluted with Tris buffer and IPA. The two toxin fractions eluted from HIC were pooled and the conductivity adjusted to 9 mS or less using WFI. The C. difficile toxins (in pooled elutate) were further purified by anion exchange chromatography. The eluted aqueous solution was passed through an anion exchange column (e.g., DEAE FF Sepharose) to bind toxins to column. The column was equilibrated with Tris buffer (50 mM Tris/50 mM NaCl pH 7.5) and toxin A was eluted with a low salt Tris buffer (141 mM NaCl/50 mM Tris, pH 7.5) and toxin B was eluted with a high salt Tris buffer (488 mM NaCl/50 mM Tris, pH 7.5). Purified toxin A and purified toxin B were each concentrated and diafiltered into 100 mM PO₄, pH 7. The steps (e.g., clarification, TFF, HIC, AEX) were each performed at ambient temperature (i.e., about 37° C.-26° C.). Average yield of toxin A was about 0.0075 g pure toxin/L fermentation and purity as evaluated by SDS Page was about 92.8% on average. Average yield of toxin B was about 0.0035 g pure toxin/L fermentation and purity as evaluated by SDS Page was about 91% on average. The concentration of proteins was determined by UV absorbance at 280 nm using absorbance units.

Inactivation of Toxins

Toxins A and B were inactivated by treatment with formaldehyde. A 37% formaldehyde solution was added aseptically to each of the Toxin A diafiltrate and the Toxin B diafiltrate to obtain a final concentration of 0.42%. The solutions were mixed and then stored at 2-8° C. for 18-22 days. Following inactivation, the toxin diafiltrates were dialyzed into formulation buffer.

### Example 2

The experiments described herein were performed to improve scalability, yield and purity of purification method. C. difficile working seed (strain VPI10463/ATCC43255) was used to inoculate preconditioned culture medium (comprising soy peptone, yeast extract, phosphate buffer and D-sorbitol, pH 7.1-7.3) in a sterile disposable bag and culture was incubated at 35-39° C. until target OD was achieved. It is noted that the inclusion of sorbitol in the preconditioned culture medium was found to significantly increase yield. The 30 L Seed 1 culture was used to inoculate culture medium in a 250 L sterile disposable culture bag and culture was incubated at 35-39° C. until target OD is achieved. The Seed 2 culture was used to inoculate 1000 L sterile disposable culture bags and culture was incubated at 35-39° C. until target OD is achieved. The culture from one more production fermentors was harvested and subjected to depth filtration (e.g., using a Pall Stax depth filter) to remove C. difficile cells and cell debris impurities and simultaneously cooled (e.g., about 37° C.-19° C.) to limit protease activity. The resulting clarified culture filtrate was concentrated and diafiltered by tangential flow filtration using flat stock Millipore (which is preferable for scale-up) and at a temperature of about 4° C. to 10° C. (for reduced protease activity) into 25 mM Tris/50 mM NaCl/0.2 mM EDTA, pH 7.5-8.0 (without DTT). The resulting solution was filtered using a membrane filter, the concentration of ammonium sulfate was increased (e.g., to about 0.4M) and then a further filtration was performed (e.g., using a membrane filter). This aqueous solution contained C. difficile toxin A and toxin B. This fermentation process (which utilizes media with sorbitol) yields 2-3 fold over the toxin yields obtained from the fermentation process substantially as described in Example 1 (i.e., 2-3 fold over approximately 5-9 μg/mL of Toxin A and 10-15 μg/mL of Toxin B). Culture filtrates were purified using a process substantially as described in Example 1 (as in paragraph

above). A number of impurities were evident in toxins A and B including alcohol dehydrogenase (ADH) in Toxin A. Three impurities were evident in Toxin B by SDS-PAGE analysis including a band at about 210 kDa, 85 kDa and 60 kDa which may represent HSP60 and degradation products. The process was attempted a number of times. Toxin purity was evaluated by SDS-PAGE and ranged from about 73.5 to 88.7%. Purity of Toxin A and Toxin B as calculated by measuring the main band visible by SDS-PAGE gel, is set out in Table 1, and ranged from about 73.5 to 88.7%. The major impurity for Toxin A was ADH (as seen by SDS-PAGE as a band at about 100 kDa) and a number of impurities were evident in Toxin B. Recoveries obtained at the HIC step were <50%.

TABLE 1

| Lot # | % Main Band |
|---|---|
| Toxin A lot 1A | 76.9 |
| Toxin A lot 2A | 80.3 |
| Toxin A lot 3A | 73.5 |
| Toxin A lot 4A | 78.5 |
| Toxin B lot 1B | 86.7 |
| Toxin B lot 2B | 79.9 |
| Toxin B lot 3B | 88.7 |
| Toxin B lot 4B | 84.9 |

Changes were made to the purification process as described below to, in part, address the increased impurity load resulting from the fermentation process improvement: (i) in the HIC step, elution was effected using Tris/2 mM EDTA and 7% IPA; and (ii) AEX was effected using DEAP.

Toxin Degradation

Toxin degradation has been observed particularly in Toxin B and may be an issue regarding purity. In Toxin B typical impurity bands are observed at 200kDa, 85kDa and 60kDa which correspond with bands reported by others as resulting from autoproteolysis in the presence of DTT. To evaluate the presence of proteases, in process samples from one lot were incubated at -80° C., 4° C. and 25° C. for several days. Minor banding pattern changes were noted in clarified broth and pre-HIC samples showing bands at about 200kDa and 85kDa showing up in 25° C. and 4° C. samples but not the −80° C. samples. A post-TFF2 sample of Toxin B was entirely degraded after four days incubation at room temperature suggesting an exogenous protease. Studies using post-TFF2 material suggest use of PO₄ as a diafiltration buffer may influence degradation. Post-TFF2 material from one lot was dialysed in 20 mM PO₄ buffer at different pH levels (6.0, 6.5, 7.0, 7.5, and 8.0). An additional sample was dialyzed into 20 mM Citrate, pH 7.5. Following dialysis, new bands were observed at about 200kDa and 85kDa at pHs in PO₄ buffer. Surprisingly, 20 mM citrate did not show this result, thus suggesting citrate buffer may be a useful diafiltration buffer.

Improved Purification Process

As mentioned above, the purification process was modified to improve yield and purity. Different HIC resins were screened. Culture filtrate, post clarification by depth filtration was processed on tangential flow filtration and membrane filtration, and was purified by HIC using one of several HIC supports (e.g., Phenyl low hydrophobicity, Phenyl high hydrophobicity (Phenyl Sepharose resin, (Phenyl Sepharose Fast Flow)), Butyl Sepharose, Buytl S Sepharose, Sartobind Nano Phenyl, and Fractogel Propyl (EMD Biosciences). Separation of ADH was not achieved using any of the HIC resins or membranes in these studies. Butyl S resulted in about 2× more capacity as compared to phenyl high sub(control) and significantly better recovery of toxin (about 90% vs. 50% for phenyl), based on SDS-PAGE data. Other benefits of the new process include that isopropyl alcohol is not required and the product may be collected a single peak after elution.

In one study, pre-HIC material was run on a Fractogel Propyl column (EMD Biosciences). As compared to phenyl sepharose resins, Fractogel propyl is weaker hydrophobic resin that includes an inert support (methacrylate), which has higher compressibility than sepharose (e.g., and may limit secondary binding of proteins). Use of a weaker hydrophobic resin like propyl and butyl eliminates the need to use a solvent following aqueous elution to completely strip the resin of product. Various loading conditions and linear gradient elutions were evaluated. As compared to HIC using phenyl sepharose, significantly better recovery of toxins was provided as well as some improvement in separation of impurity from toxins. Results indicated that (i) Fractogel Propyl resin may substitute for the phenyl resin; (ii) a loading condition of 1.2M ammonium sulphate concentration is sufficient to bind toxin to the propyl resin, (iii) a solvent is not required to complete the elution process like is current seen for the phenyl resin and (iv) resolution of toxins A and B may be possible using propyl resin as opposed to co-elution of both to be separated later by AEX chromatography.

In another study, HIC was performed using Butyl S Sepharose (GE Healthcare Hitrap Butyl-S). Pre-HIC material was diluted with a 1X volume of 2.5M ammonium sulfate, 50 mM Tris, pH 7.5 and run on the column as per manufacturer instructions and using a linear gradient to co-elute toxins. Post-HIC product from the butyl-S sepharose chromatography run was compared by SDS-PAGE to post-HIC product from phenyl sepharose chromatography runs. HIC chromatography using Butyl-S sepharose had significantly lower levels of molecular weight impurities. The eluted toxins from the Butyl-S HIC contained a lower ratio of impurities than material purified using phenyl sepharose. As compared to HIC using phenyl sepharose and HIC using Fractogel propyl, significantly better separation of toxins from impurities was provided by Butyl-S HIC. In one study comparing HIC/phenyl sepharose and HIC/Butyl S, only 10% recovery of Toxin A was obtained using HIC/phenyl Sepharose while HIC/Butyl S provided almost 70% recovery for Toxin A and almost 90% recovery for Toxin B using the same clarified culture filtrate starting material. Therefore, as compared to more hydrophobic phenyl Sepharose resins, less hydrophobic resins (e.g., butyl Sepharose resins such as, e.g., GE Butyl S FF Sepharose) provided significantly improved recovery at the HIC step and also permitted the elution of the toxins without the use of solvent (e.g., IPA).

A number of anion-exchange supports were also evaluated. Culture filtrate, post purification by HIC/Phenyl Sepharose or HIC/Butyl S Sepharose, was purified using one of several AEX columns. Gradient elution of 10 column volumes was used. Screening results are set out in Table 2. The ADH impurity co-eluted with Toxin A when AEX was performed using a DEAP column at each of the pHs tested and with an elution buffer with 0.5M Arginine. Use of a DEAP column with a 1M Acetate elution buffer showed little to no separation of the ADH impurity from Toxin A. AEX chromatography using a Tosoh DEAE column also showed little to no separation of the ADH impurity from Toxin A. Runs of Butyl S Sepharose eluted material purified on Tosoh Q resulted in very pure material, with Toxin A having a purity of 94% or greater. A number of runs using broth filtrate from 2000 L fermentation lots and purified using HIC with Butyl S Sepharose and AEX with Tosoh Q provided Toxin A having an average purity of 99% and Toxin B having an average purity of 98%. In regards to Toxin B, HIC using Butyl S Sepharose (as compared to Phenyl Sepharose) and AEX using Tosoh Q (as compared to DEAP) improved purity and yield. Toxin B degradation and purity were further improved with removal of DTT and replacement of phosphate buffer with a citrate buffer in the second tangential flow filtration step (although the phosphate buffer is typically preferred for other reasons). Therefore, good separation of one impurity (alcohol dehydrogenase (ADH)) from toxin A was seen with non-polysaccharide based supports (such as for example, methacrylate based resins, polyethersulfone based membranes). Good separation of toxin B from other impurities such as HSP-60 and other proteins was also observed. In comparison to the DEAE-Sepharose resin, these non-polysaccharide based supports increased toxin purity and yield, as shown in Table 2.

TABLE 2

| | | | Yields | Purity |
|---|---|---|---|---|
| | | | Toxin A/ | Toxin A/ |
| AEX Support | Type | ADH | Toxin B | Toxin B |
| Tosoh | Methacrylate based | Good | >70% for | 99% |
| Toyopearl | resin with | separation | Toxin A | Toxin A/ |
| Super Q 650 | quaternary | | and B | 98% |
| (Tosoh | ammonium | | | Toxin B |
| Bioscience | functionality | | | from |
| LLC)* | attached to provide | | | 2000 L |
| | a positive charge | | | runs |
| | over a broad pH | | | |
| | range | | | |
| Tosoh | Methacrylate based | Moderate | >70% for | less than |
| Toyopearl | resin with DEAE | separation | Toxin A | 99% |
| DEAE-650 | functional group. | | and B | Toxin A/ |
| (Tosoh | A weak anion | | | 98% |
| Bioscience | exchanger | | | Toxin B |
| LLC)° | | | | |
| Elution buffer: | | | | |
| MgCl₂ in Tris | | | | |
| Natrix Q | Microporous | Good | <70% for | similar to |
| membrane | polyethersulfone | separation | Toxin A | tosoh |
| (Natrix | (PES) membrane | | and B | Super Q |
| Separations, | with quaternary | | | |
| Inc.)° | amine functional | | | |
| | groups | | | |
| Mustang-Q | Microporous | Good | >70% for | lower |
| membrane | polyethersulfone | separation | Toxin A | than |
| (Pall | (PES) membrane | | and B | tosoh |
| Corporation)° | with quaternary | | | super Q |
| | amine functional | | | and not |
| | groups | | | quite as |
| | | | | good a |
| | | | | natirx |
| Bio-Rad | Methacrylate based | Good | >70% for | 88% (A) |
| Macro-Prep | resin (methacrylate | separation | Toxin A | |
| High Q (Bio- | copolymer bead) | | and B | |
| Rad)° | with functional | | | |
| | group, N(CH₃)₃. A | | | |
| | strong anion | | | |
| | exchanger. | | | |
| Sartorius Q | Glucose-based | Poor | <70% for | lower |
| membrane | resin with | separation | Toxin A | purity |
| (Sartorius)° | quaternary and | | and B | than |
| | secondary amine | | | super Q |
| | functional groups | | | |
| | Sartobind ® | | | |
| | capsules with 4 mm | | | |
| | bed height with ion | | | |
| | exchange | | | |
| | membranes of | | | |
| | stabilised | | | |
| | reinforced cellulose | | | |
| | with Quaternary | | | |
| | ammonium | | | |
| | functional group | | | |
| | A strong anion | | | |
| | exchanger | | | |
| GE DEAE | Glucose-based | Poor | >70% for | 50-80% |
| Sepharose (GE | resin with | separation | Toxin A | |
| Healthcare)° | quaternary and | | and B | |
| | secondary amine | | | |
| | functional groups | | | |
| | Matrix is 6% cross- | | | |
| | linked agarose and | | | |
| | DEAE functional | | | |
| | group. A weak | | | |
| | anion exchanger. | | | |
| GE DEAP | Glucose-based | Poor | 52.3% for | 50-70% |
| (Diethyl | resin with | separation | Toxin A/ | |
| aminopropyl) | quaternary and | of impurity | 17.7% for | |
| Sepharose | secondary amine | | Toxin B | |
| (ANX | functional groups | | | |
| Sepharose Fast | | | | |
| Flow, GE | | | | |
| Healthcare) | | | | |
| Elution buffer: | | | | |
| MgCl₂ in Tris, | | | | |
| pH 6.5° | | | | |
| GE DEAP | Glucose-based | No | >70% for | 50-70% |
| (Diethyl | resin with | separation | Toxin A | |
| aminopropyl) | quaternary and | | and B | |
| Sepharose | secondary amine | | | |
| (ANX | functional groups | | | |
| Sepharose Fast | | | | |
| Flow, GE | | | | |
| Healthcare), | | | | |
| Elution buffer: | | | | |
| MgCl₂ in Tris, | | | | |
| pH 7.5° | | | | |
| GE DEAP | Glucose-based | No | >70% for | 50-70% |
| (Diethyl | resin with | separation | Toxin A | |
| aminopropyl) | quaternary and | | and B | |
| Sepharose | secondary amine | | | |
| (ANX | functional groups | | | |
| Sepharose Fast | | | | |
| Flow, GE | | | | |
| Healthcare), | | | | |
| Elution buffer: | | | | |
| MgCl₂ in Tris, | | | | |
| pH 8.5° | | | | |
| GE DEAP | Glucose-based | No | >70% for | 50-70% |
| (Diethyl | resin with | separation | Toxin A | |
| aminopropyl) | quaternary and | | and B | |
| Sepharose | secondary amine | | | |
| (ANX | functional groups | | | |
| Sepharose Fast | | | | |
| Flow, GE | | | | |
| Healthcare), | | | | |
| Elution buffer: | | | | |
| 0.5M | | | | |
| Arginine/Tris | | | | |
| pH 7.5° | | | | |
| GE DEAP | Glucose-based | Little to no | >70% for | 50-70% |
| (Diethyl | resin with | separation | Toxin A | |
| aminopropyl) | quaternary and | | and B | |
| Sepharose | secondary amine | | | |
| (ANX | functional groups | | | |
| Sepharose Fast | | | | |
| Flow, GE | | | | |
| Healthcare), | | | | |
| Elution | | | | |
| Buffer: 1M | | | | |
| acetate/Tris, | | | | |
| pH 7.5° | | | | |
| GE Q | Glucose-based | Poor | <70% for | 50-70% |
| Sepharose Fast | resin with | separation | Toxin A | |
| Flow (GE | quaternary and | | and B | |
| Healthcare)° | secondary amine | | | |
| | functional groups | | | |
| | Matrix is 6% | | | |
| | spherical, cross- | | | |
| | linked agarose with | | | |
| | quaternary amine | | | |
| | group functional | | | |
| | group | | | |
| | (—Ch₂N⁺(CH₃)₃, | | | |
| | quaternary | | | |
| | ammonium) | | | |
| | A strong anion | | | |
| | exchanger. | | | |
| Fractogel ® | A synthetic | Good | 33.85% for | 94.9% |
| EMD TMAE | methacrylate based | separation | Toxin A | for Toxin |
| (EMD | polymeric resin | | 41.54% for | A |
| Millipore)° | with quaternary | | Toxin A | ND |
| | amine functional | | 13.6% for | ND |
| | groups | | Toxin B | |
| | Strong anion | | | |
| | exchanger | | | |

°HIC performed using Phenyl Sepharose
*HIC performed using Butyl S Sepharose
ND² yield not calculated due to issues during sample loading

Two purification procedures were compared using 20 L filtration broth from C. difficile cultured substantially as described in this Example: (i) the first used Butyl HIC Sepharose for the HIC step and Tosoh Q resin for the AEX step; and, (ii) the second used Phenyl HIC Sepharose for the HIC step and DEAP Sepharose for the AEX step. Purity was evaluated by SDS-PAGE. Toxin A and B purified by the first process had a purity of >97% and 95%, respectively. Following storage at 25° C. for one week, these toxins showed good stability and had no change in purity. Toxins were stable even in buffers without citrate. Toxin purity using the second process was less (e.g., 76% for Toxin A and 28% for Toxin B). The stability of these toxins following storage for one week at 4° C. was poor.

Order of HIC and AEX

The order of HIC and AEX chromatography steps was alternated to evaluate the effects on yield and purity. Clarified harvest was first purified by AEX chromatography (using a Tosoh Q resin) and the resulting Toxin A and Toxin B products were further purified by HIC chromatography (using a Fractogel Propyl resin). Using a linear gradient, two elution peaks with base line resolution were observed from the AEX column. Analysis by SDS-PAGE confirmed that the first elution peak corresponded to Toxin A and the second elution peak corresponded to Toxin B. The contaminant profile for each peak was significantly different. The fractions from each peak were pooled to create a Toxin A and a Toxin B starting material for further purification by HIC. One major elution peak was observed for each HIC separation, and the peak fractions when analyzed by SDS-PAGE showed predominantly Toxin A or Toxin B. The results from this study show (i) Toxins A and B from clarified harvest can be separated to base line resolution, and (ii) the individual toxins can be further purified and polished on the propyl resin.

HIC using Butyl S Sepharose and AEX using Tosoh Q

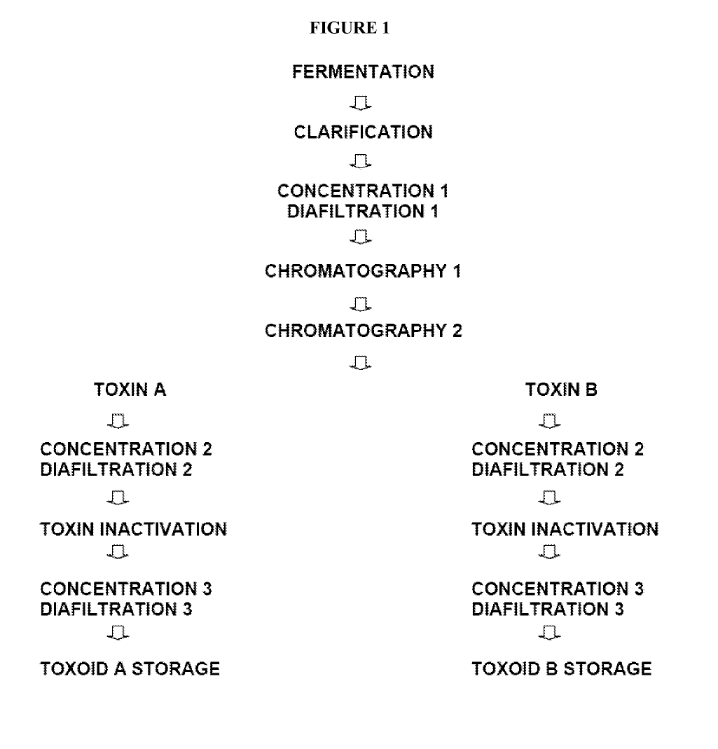

A purification process using Butyl HIC Sepharose for the HIC step and Tosoh Q resin for the AEX step was established (FIG. 1). Multiple large-scale runs (160 L or greater) were performed. Purity as evaluated by SDS PAGE was, on average, about 98% and 97% for Toxin A and B, respectively. Average yields were 0.024 and 0.12 g toxin /L fermentation broth (e.g., containing sorbitol) for Toxin A and B, respectively, which represents a 3-5 fold increase over yields obtained using the process described in Example 1. Similar yields and purity results were obtained in 1000 L and 2000 L runs. Therefore, substituting phenyl sepharose with Butyl S for HIC and substituting DEAP with Tosoh Q for AEX provides good purity and recovery of Toxin A and Toxin B (in part due to the inclusion of sorbitol in the culture medium). Subsequent studies using Butyl S for HIC, and Tosoh Q for AEX conducted without any addition of DTT to sample or buffers and using MgCl₂ in AEX elution buffer, further improved purity and decreased toxin degradation.

Inactivation of Toxins

Toxins A and B purified as described above were inactivated by treatment with formaldehyde. A 37% formaldehyde solution was added aseptically to each of the Toxin A diafiltrate and the Toxin B diafiltrate to obtain a final concentration of about 0.2%-0.4%. The solutions were mixed and then stored at about 25° C. for 6 to 13 days. Following inactivation, the toxin diafiltrates were dialyzed into formulation buffer and assayed in vivo as described in Example 3.

### Example 3

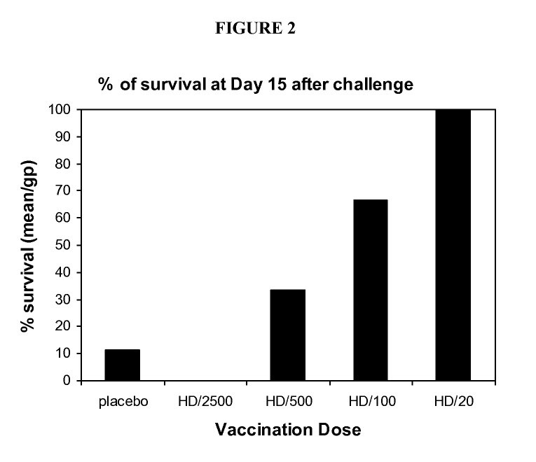

Purified C. difficile Toxoid A and C. difficile Toxoid B were prepared substantially in accordance with the methods set out in Example 2 and formulated as vaccine compositions. Toxoids A and B were combined at a ratio of 3:2 by weight, formulated with a citrate buffer comprising sucrose (4.0% to 8.0% w/v) and formaldehyde (0.012% to 0.020% w/v) and lyophilized. Each composition was reconstituted with diluent and adjuvanted with aluminum hydroxide prior to use as a vaccine. Syrian gold hamsters provide a stringent model for C. difficile vaccine development. After being pretreated with a single intraperitoneal (IP) dose of clindamycin antibiotic and after receiving an intragastric (IG) inoculation of toxigenic C. difficile organisms, the hamsters rapidly develop fulminant diarrhea and hemorrhagic cecitis and die within two to four days (e.g., without vaccination). The reconstituted vaccine contained 100 μg/dose toxoids, 0.008% formaldehyde and 400 μg/dose aluminum. Hamsters (9 hamsters/group) were vaccinated by three intramuscular immunizations (at Day 0, Day 14, and Day 27) with different doses of C. difficile vaccine (4 dilutions of human dose (100 μg/dose) (HD) or were injected with the placebo (A1OH). At Day 41, hamsters were pretreated with chemical form of Clindamycin-2-phosphate antibiotic at 10 mg/kg by IP route. At Day 42, after 28 hours pretreatment with antibiotic, hamsters were challenged by IG route with a lethal dose of spore preparation derived from C. difficile ATCC43255 strain. The protective efficacy was assessed by measuring the kinetics of apparition of symptoms associated with C. difficile infection and lethality. Results demonstrated that the vaccine protects hamsters against lethal challenge with C. difficile toxigenic bacteria in a dose-dependent manner, with 100% protection induced by vaccination with the dose HD/20 (5 μg Toxoid A+B in presence of 100 μg/mL AlOH) (FIG. 2). Immunized animals were protected against death and disease (weight loss and diarrhea). The results of this study are representative of several in vivo studies. Accordingly, toxoids prepared by the disclosed methods provide protective immunity against C. difficile disease.

While certain embodiments have been described in terms of the preferred embodiments, it is understood that variations and modifications will occur to those skilled in the art. Therefore, it is intended that the appended claims cover all such equivalent variations that come within the scope of the following claims.

1. A method for the production of purified C. difficile toxin, the method comprising:
a) applying an impure aqueous solution comprising C. difficile toxin to a hydrophobic interaction support to bind C. difficile toxin thereto;
b) eluting the bound C. difficile toxin from the hydrophobic interaction support;
c) applying the eluted C. difficile toxin from step (b) to an anion-exchange support selected from the group consisting of: (i) a polymethacrylate resin with quaternary amine functional groups; and (ii) a polyethersulfone membrane with quaternary amine functional groups, to bind C. difficile toxin thereto; and,
d) eluting the bound C. difficile toxin from the anion-exchange support.

2. The method of claim 1 further comprising subjecting the impure aqueous solution comprising C. difficile toxin to tangential flow filtration before step (a).

3. The method of claim 1 further comprising recovering purified C. difficile toxin following step (d).

4. The method of claim 1 wherein the toxin is C. difficile toxin A.

5. The method of claim 1 wherein the toxin is C. difficile toxin B.

6. The method of any one of claims 14 wherein the hydrophobic interaction support is a matrix with attached butyl S groups or a matrix with attached propyl groups.

8. The method of claim 6 wherein the hydrophobic interaction support is a matrix with attached propyl groups and the impure aqueous solution is applied to the hydrophobic interaction support in the presence of ammonium sulphate.

9. The method of claim 8 wherein the impure aqueous solution is applied to the hydrophobic interaction support in the presence of about 0.8 to about 1.0M ammonium sulphate.

10. The method of claim 1 wherein step (b) is performed in the absence of an organic solvent.

11. The method of claim 6 wherein the hydrophobic interaction support is a Sepharose resin with attached butyl S groups.

12. The method of claim 6 wherein about 70% of the toxin A and/or about 90% of the toxin B in the impure aqueous solution is recovered after steps (a) and (b).

13. The method of claim 6 wherein step (b) is performed in the absence of an organic solvent.

14. The method of claim 1 wherein the aqueous solution comprising C. difficile toxin comprises C. difficile toxin A and C. difficile toxin B and, in step (c), C. difficile toxin A and C. difficile toxin B are individually eluted from the anion-exchange support.

15. The method of claim 1 wherein the anion exchange support is a non-polysaccharide-based material.

16. The method of any one of claims 15 wherein the anion exchange support is a polymethacrylate resin with bound quaternary amine functional groups.

17. The method of any one of claims 16 wherein the anion exchange support comprises a strong anion exchanger, the moiety —O—R—N⁺—(CH₃)₃ wherein R is a polymer; and/or, the anion exchange support is Tosoh Super Q 650M.

18. The method of claim 14 wherein toxin A is eluted using a low salt buffer.

19. The method of claim 19 wherein toxin A the low salt buffer comprises 2 to about 32 mM MgCl₂ or about 125 to about 160 mM NaCl.

20. The method of claim 14 wherein toxin B is eluted using a high salt buffer.

21. The method of claim 20 wherein the high salt buffer comprises about 120 to about 150 mM MgCl₂ or about 450 to about 550 mM NaCl.

22. The method of claim 1 wherein the impure aqueous solution of C. difficile toxin is obtained by growing cells of C. difficile in a growth medium to provide a culture broth containing C. difficile toxin and grown cells, and separating the culture broth from the grown cells to provide the impure aqueous solution of C. difficile toxin.

23. The method of claim 22 wherein the growth medium comprises sorbitol.

24. A purified C. difficile toxin obtained in accordance to the method of claim 1 wherein the purified C. difficile toxin has a purity of about 90% or greater.

25. The purified C. difficile toxin of claim 24 wherein the toxin has a purity of about 94% or greater.

26. The purified C. difficile toxin of claim 25 wherein the toxin has a purity of about 98% or greater.

27. The purified C. difficile toxin A of claim 26 having a purity of about 99% or greater.

28. The purified C. difficile toxin of claim 24 wherein the toxin is C. difficile Toxin A.

29. The purified C. difficile toxin of claim 24 wherein the toxin is C. difficile Toxin B.

30. A purified C. difficile toxin obtained in accordance to the method of claim 1 wherein the purified C. difficile toxin is substantially free of solvent.

31. A purified C. difficile toxin obtained in accordance to claim 1 wherein the purified C. difficile toxin has no detectable alcohol dehydrogenase.

32. The method of claim 1 further comprising inactivating recovered purified C. difficile toxin with a chemical agent to provide a C. difficile toxoid.

33. The method of claim 32 wherein the recovered purified C. difficile toxin is inactivated with formaldehyde to provide a C. difficile toxoid.

34. A C. difficile toxoid obtained in accordance to the method of claim 32.

35. The C. difficile toxoid of claim 34 wherein the toxoid is toxoid A or toxoid B.

36. A composition comprising the C. difficile toxoid of claim 34.

37. A composition comprising the C. difficile toxoid of claim 34 and a pharmaceutically acceptable excipient.

38. The composition of claim 36 wherein the C. difficile toxoid is toxoid A or toxoid B.

39. The composition of claim 36 comprising C. difficile Toxoid A and C. difficile B at a ratio of 3:2 by weight.

40. A method of eliciting an immune response in a subject, the method comprising administering to the subject a composition of claim 36.

41. A method of preventing or treating symptomatic C. difficile infection in a subject, the method comprising administering to the subject a composition of claims 36.

42. The method of claim 41 wherein the subject does not have symptoms of but is at risk of developing symptomatic C. difficile infection.

43. The method of claim 41 wherein the subject has symptomatic C. difficile infection.
